(19) United States
(12) Patent Application Publication
(10) Pub. No.: US 2019/0127515 A1
(43) Pub. Date: May 2, 2019

(54) KITS FOR MAKING RECYCLABLE COVALENTLY CROSSLINKED POLYMER NETWORKS, RECYCLABLE COMPOSITES, AND RELATED METHODS OF MAKING RECYCLABLE COMPOSITES AND RECYCLING COMPOSITES

(71) Applicant: Western Washington University, Bellingham, WA (US)

(72) Inventors: John Misasi, Bellingham, WA (US); Scott Lew, Bellingham, WA (US)

(73) Assignee: Western Washington University, Bellingham, WA (US)

(21) Appl. No.: 16/172,572

(22) Filed: Oct. 26, 2018

Related U.S. Application Data
(60) Provisional application No. 62/577,576, filed on Oct. 26, 2017.

(51) Int. Cl. C08G 59/50; C07D 251/04; C08G 59/24; C08G 59/28; C08G 18/10; C08G 18/38; C08K 3/04; C08J 11/10; C08K 7/06

(52) U.S. Cl. CPC C08G 59/5086; C07D 251/04; C08G 59/245; C08G 59/28; C08G 18/10; C08J 2367/00; C08K 3/04; C08J 11/10; C08K 7/06; C08J 2363/00; C08J 2375/04; C08G 18/3851

(57) ABSTRACT

Kits, recyclable covalently crosslinked polymer networks and composites, and related methods of making and recycling are described.

### CROSS-REFERENCE(S) TO RELATED APPLICATION(S)

This application claims the benefit of the U.S. Provisional Patent Application No. 62/577,576 filed Oct. 26, 2017, the disclosure of which is incorporated herein by reference in its entirety.

### BACKGROUND

As the demand and consumption of technology increases, so does the burgeoning need for our society to embrace reusable, recyclable materials. However, many of the materials used in high-technology industries, like electronics, transportation, and construction, are not recyclable. One such family of materials has been glassy polymer networks. Glassy polymer networks are commercially relevant materials that are utilized in industries ranging from aerospace composites to electronics. The thermosetting nature of polymer networks provides ease of processing, high modulus, low solvent and moisture ingress, and other properties that are suitable for demanding applications. However, once network formation has completed (past gel and/or to full conversion) the material becomes intractable and does not melt upon heating, thus recycling of infinite networks into either similar or dissimilar starting materials becomes impossible.

Studies to improve the recyclability of thermoset networks have shown promise, with many new materials being developed that incorporate functionalities and mechanisms for network degradation, re-workability, and recyclability. One such material family is covalent adaptive networks (CANs). CANs use reversible crosslinking and de-crosslinking of carbon-carbon bonds within a network to produce semi-solid, viscous polymers when stimulated. While many adaptive networks are promising materials, one of the primary challenges to their successful commercial implementation is meeting the thermal stability requirements of high-temperature applications where glass transition temperatures above 100° C. are common.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the present disclosure provides a kit including an amine curative agent having a pH-responsive chemical linkage and a prepolymer. Recyclable networks made from the kits described herein include pH-degradable covalent networks that have high glass transition temperatures, such as over 100° C., have high mechanical stability, and are recyclable under moderately acidic conditions, such as conditions having a pH of 2.5 or less.

In an embodiment, the kits of the present disclosure comprise a curative agent comprising a hexahydrotriazine moiety and a prepolymer, wherein when reacted the curative agent and the prepolymer react to form a covalently crosslinked polymer network that is degradable in acidic conditions.

In another aspect, the present disclosure provides a curative agent including a hexahydrotriazine moiety.

In another aspect, the present disclosure provides a covalently crosslinked polymer network comprising a reaction product of the kits described herein.

In another aspect, the present disclosure provides a composite comprising the covalently crosslinked polymer networks described herein; and a plurality of fibers disposed within the covalently crosslinked polymer network.

In another aspect, the present disclosure provides a method of making a covalently crosslinked polymer network comprising: providing a mixture comprising: a curative agent comprising a pH-responsive chemical linkage; and a pre-polymer; and heating the mixture to form a covalently crosslinked polymer network degradable under acidic conditions.

In an embodiment, the curative agent includes a hexahydrotriazine moiety.

In another aspect, the present disclosure provides a method of recycling a covalently crosslinked polymer network comprising: exposing the covalently crosslinked polymer network to acidic conditions, thereby degrading the covalently crosslinked polymer network, wherein the covalently crosslinked polymer network comprises: a reaction product of a prepolymer and a curative agent comprising a pH-responsive chemical linkage.

### DESCRIPTION OF THE DRAWINGS

The foregoing aspects and many of the attendant advantages of the claimed subject matter will become more readily appreciated as the same become better understood by reference to the following detailed description, when taken in conjunction with the accompanying drawings, wherein:

FIG. 1 illustrates a representative ¹H-NMR spectrum of a 1,3,5-hexahydrotriazine triamine (xTAZ) curative agent and chemical moieties of the curative agent corresponding to portions of the ¹H-NMR spectrum (inset), in accordance with an embodiment of the disclosure;

FIG. 2 graphically illustrates a characteristic Fourier transform infrared spectroscopy (FTIR) spectrum of an xTAZ curative agent, in accordance with an embodiment of the disclosure;

FIG. 3 graphically illustrates a representative cure exotherm and ultimate glass transition temperature of a covalently crosslinked polymer network, in accordance with embodiments of the disclosure, according to differential scanning calorimetry (DSC) measurements of first (dashed) and second (solid) thermal cycles from single heat/cool/heat experiment;

FIG. 4 graphically illustrates thermo-mechanical properties, storage modulus E′ (dashed) and tan δ (solid), of a recyclable xTAZ covalently crosslinked polymer network, in accordance with an embodiment of the disclosure, by dynamic mechanical analysis (DMA);

FIG. 5 graphically illustrates (right) acid ingress in recyclable xTAZ-diglycidylether of bisphenol A (DGEBA) covalently crosslinked polymer network at various temperatures, in accordance with an embodiment of the disclosure; and (left) a comparison of acid ingress rates in xTAZ-DGEBA networks in accordance with embodiments of the disclosure at three different temperatures;

FIG. 6 is (left) a scanning electron microscopy (SEM) image of neat carbon fibers; and (right) an SEM image of recycled carbon fibers showing little residual matrix and/or fiber damage, in accordance with embodiments of the disclosure;

FIG. 7 graphically illustrates a FTIR comparison of xTAZ-DGEBA networks (dashed line), in accordance with embodiments of the disclosure, and recycled polymer products (solid line);

FIG. 8 graphically illustrates ultimate glass transition temperatures of reversible epoxy network (xTAZ, dashes), in accordance with embodiments of the disclosure, and comparative network (xylylene diamine, solid line);

FIG. 9 graphically illustrates thermogravimetric analysis (TGA) thermograms of reversible epoxy (xTAZ, dashed line), in accordance with an embodiment of the disclosure, and comparative network (xylylene diamine, solid line); and

FIG. 10 graphically illustrates a comparison of storage modulus as a function of temperature obtained via DMA of a covalently crosslinked polymer network (xTAZ, dashed line), in accordance with an embodiment of the disclosure, and neat network (xylylene diamine, solid line); and

FIG. 11 schematically illustrates a twin-screw extruder for use in preparing curative agents, in accordance with an embodiment of the disclosure.

### DETAILED DESCRIPTION

The detailed description set forth below in connection with the appended drawings is intended as a description of various embodiments of the claimed subject matter and is not intended to represent the only embodiments. Each embodiment described in this disclosure is provided merely as an example or illustration and should not be construed as preferred or advantageous over other embodiments. The illustrative examples provided herein are not intended to be exhaustive or to limit the claimed subject matter to the precise forms disclosed.

The present disclosure generally relates to kits; covalently crosslinked polymer networks, such as thermosets, including a reaction product of kits; composites including covalently crosslinked polymer networks and a plurality of fibers; and methods of making and recycling covalently crosslinked polymer networks. Generally described, conventional thermosets form irreversibly, permanently entraining fibers disposed therein. While certain thermosets have been shown to degrade in the presence of acidic conditions, many such thermosets have not had glass transition temperatures above, for example, 100° C., suitable for many currently used manufacturing processes. Further, certain recyclable thermosets require solvent processing and strong acids to degrade covalently crosslinked polymer networks made with such thermosets, thus limiting their applicability. Additionally, certain recyclable thermosets include highly flexible moieties that have lower chemical and structural tailorability.

To that end, the following discussion provides examples of, inter alia, kits including curative agents having pH-reversible hexahydrotriazine moieties. As discussed in more detail below, such curative agents are configured to react with a prepolymer to provide covalently crosslinked polymer networks that have high glass transition temperatures, have high mechanical stability, and are recyclable under moderately-acidic conditions.

In the following description, numerous specific details are set forth in order to provide a thorough understanding of one or more embodiments of the present disclosure. It will be apparent to one skilled in the art, however, that many embodiments of the present disclosure may be practiced without some or all of the specific details. In some instances, well-known process steps have not been described in detail in order not to unnecessarily obscure various aspects of the present disclosure. Further, it will be appreciated that embodiments of the present disclosure may employ any combination of features described herein.

In an aspect, the present disclosure provides a kit comprising a curative agent comprising a pH-responsive chemical linkage and a prepolymer, wherein when reacted the curative agent and the prepolymer react to form a covalently crosslinked polymer network that is degradable in acidic conditions. In an embodiment, the curative agents described herein include any curative agent including a hexahydrotriazine moiety configured to react with a prepolymer to form a covalently crosslinked polymer network. As used herein, a pH-responsive chemical linkage refers to a bond or other coupling that breaks or changes in response to a change in pH. In an embodiment, such a pH-responsive linkage includes a bond that breaks in response to acidic conditions. In an embodiment, the pH-responsive linkage includes a bond configured to break in response to conditions having a pH lower than, for example, about 2.5.

In an embodiment, the curative agent has the structure:

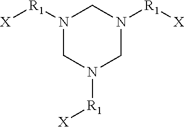

wherein,

each R₁ is independently selected from the group consisting of alkyl, alkenyl, alkynl, sulfone, and aryl groups, and

each X is independently selected from a hydroxyl group, a primary amine, and a secondary amine.

In an embodiment, X is a primary amine. As discussed further herein, in certain embodiments, the two amine hydrogens of such primary amine are each configured to react with a prepolymer, thereby providing additional crosslinks to a covalently crosslinked polymer network over a curative agent having, for example, a single amine hydrogen.

In an embodiment, the curative agent has the structure:

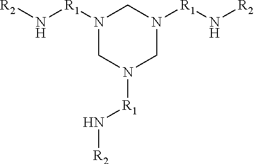

wherein,

each R₁ is independently selected from the group consisting of alkyl, alkenyl, alkynl, sulfone, and aryl groups, and

each R₂ is independently selected from the group consisting of H, alkyl, alkenyl, alkynl, and aryl groups.

In an embodiment, the curative agent is selected from the group consisting of:

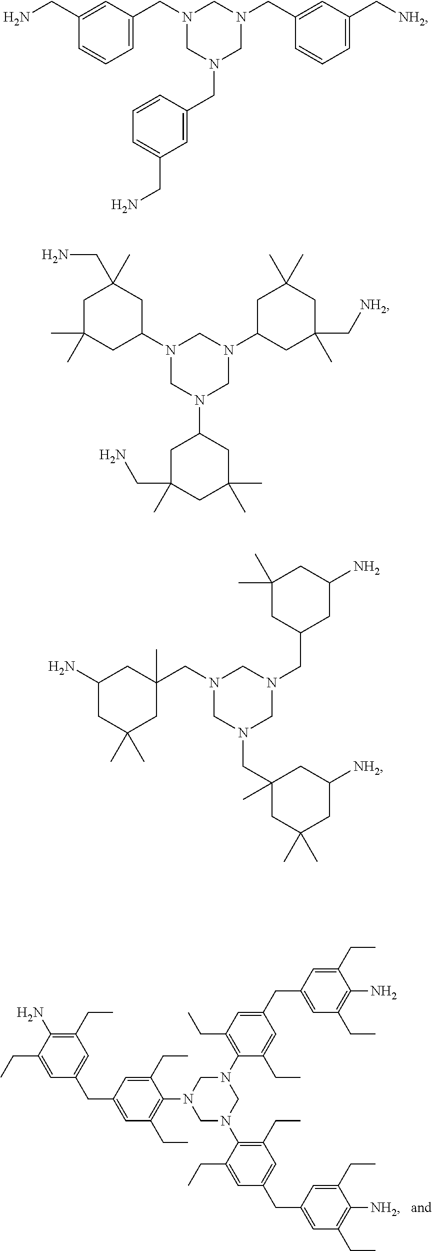

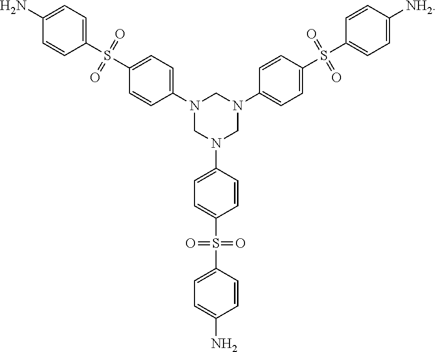 [SMILES: Nc1ccc(S(=O)(=O)c2ccc(N3CN(c4ccc(S(=O)(=O)c5ccc(N)cc5)cc4)CN(c4ccc(S(=O)(=O)c5ccc(N)cc5)cc4)C3)cc2)cc1]

In an embodiment, the curative agent is

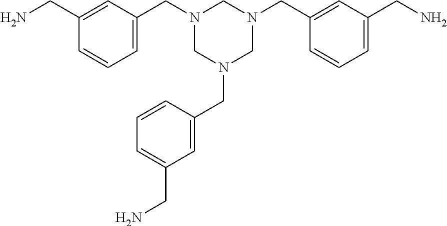 [SMILES: NCc1cccc(CN2CN(Cc3cccc(CN)c3)CN(Cc3cccc(CN)c3)C2)c1]

The kits described herein include a prepolymer configured to react with curative agents to form covalently crosslinked polymer networks. The prepolymer can be any prepolymer configured to react with a curative agent to form a covalently crosslinked polymer network configured to degrade under acidic conditions. In an embodiment, the prepolymer comprises a diisocyanate. In an embodiment, the prepolymer comprises a diester.

In certain embodiments, the prepolymer comprises an epoxy. In an embodiment, the epoxy has the structure:

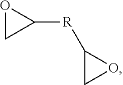

wherein R is selected from the group consisting of alkyl, aryl, alkenyl, and alkynl groups.

In an embodiment, the epoxy has the structure

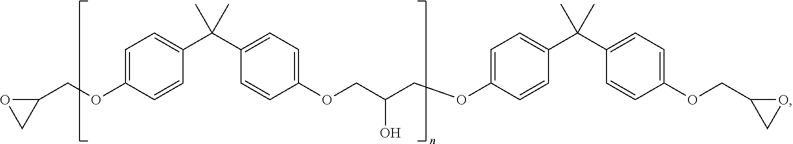

wherein

n is an integer between 1 and 5.

In an embodiment, the epoxy is selected from the group consisting of:

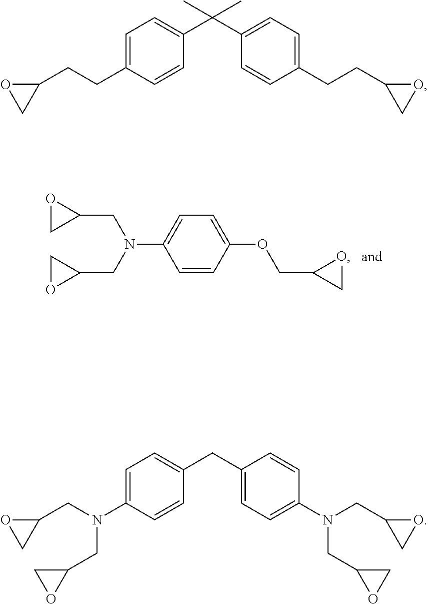 [SMILES: CC(C)(c1ccc(CCC2CO2)cc1)c1ccc(CCC2CO2)cc1 ; c1cc(N(CC2CO2)CC2CO2)ccc1OCC1CO1 ; "and" ; c1cc(N(CC2CO2)CC2CO2)ccc1Cc1ccc(N(CC2CO2)CC2CO2)cc1]

In an embodiment, the kit includes a plurality of fibers. As discussed further herein, when the components of the kit react to form a covalently crosslinked polymer network around such fibers a composite structure is formed including the covalently crosslinked polymer network and fibers disposed therein. In an embodiment, the plurality of fibers include fibers selected from the group consisting of carbon fibers, glass fibers, aramid fibers, hemp fibers, sisal fibers, coconut fibers, flax fibers, and combinations thereof.

In another aspect, the present disclosure provides a covalently crosslinked polymer network comprising a reaction product of a prepolymer and a curative agent comprising a triazine moiety. In an embodiment, the curative agent includes a hexahydrotriazine moiety.

In an embodiment, the curative agent is any curative agent described herein. In an embodiment, the prepolymer is any prepolymer described herein.

In an embodiment, the curative agent has the structure:

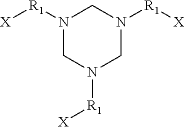

wherein,

each R₁ is independently selected from the group consisting of alkyl, alkenyl, alkynl, sulfone, and aryl groups, and

each X is independently selected from a hydroxyl group and an amine.

In an embodiment, the curative agent has the structure:

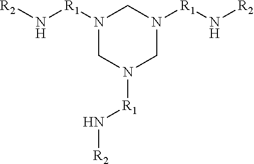

wherein,

each R₁ is independently selected from the group consisting of alkyl, alkenyl, alkynl, sulfone, and aryl groups, and

each R₂ is independently selected from the group consisting of H, alkyl, alkenyl, alkynl, and aryl groups.

In an embodiment, the prepolymer includes one or more epoxide moieties and the curative agent includes one or more amine hydrogens, and wherein the molar ratio of epoxide moieties to amine hydrogens is in a range of about 1:1 to about 1:2.

In an embodiment, the covalently crosslinked polymer network comprises a crosslinked moiety having the structure:

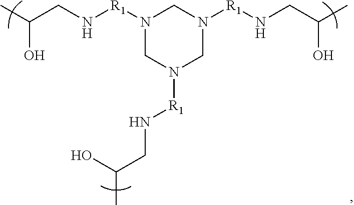

wherein each R1 is independently selected from the group consisting of alkyl, alkenyl, alkynl, sulfone, and aryl groups.

In an embodiment, the covalently crosslinked polymer network comprises a crosslinked moiety having the structure:

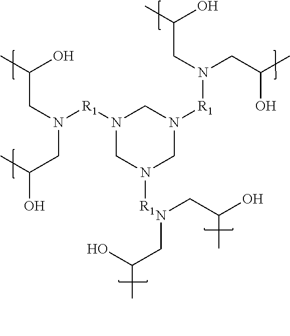

wherein each R1 is independently selected from the group consisting of alkyl, alkenyl, alkynl, sulfone, and aryl groups.

In embodiment, the covalently crosslinked polymer network comprises a crosslinked moiety having the structure:

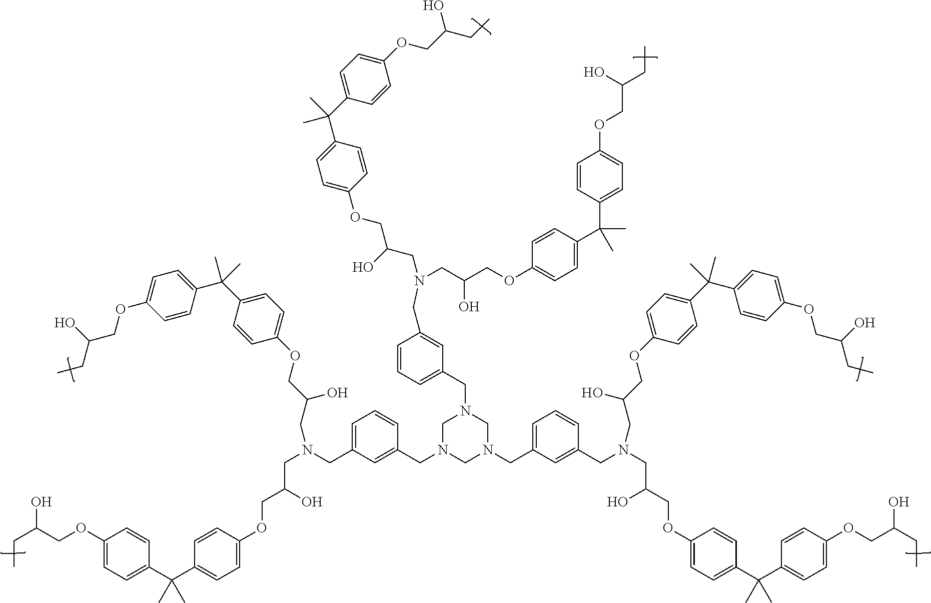

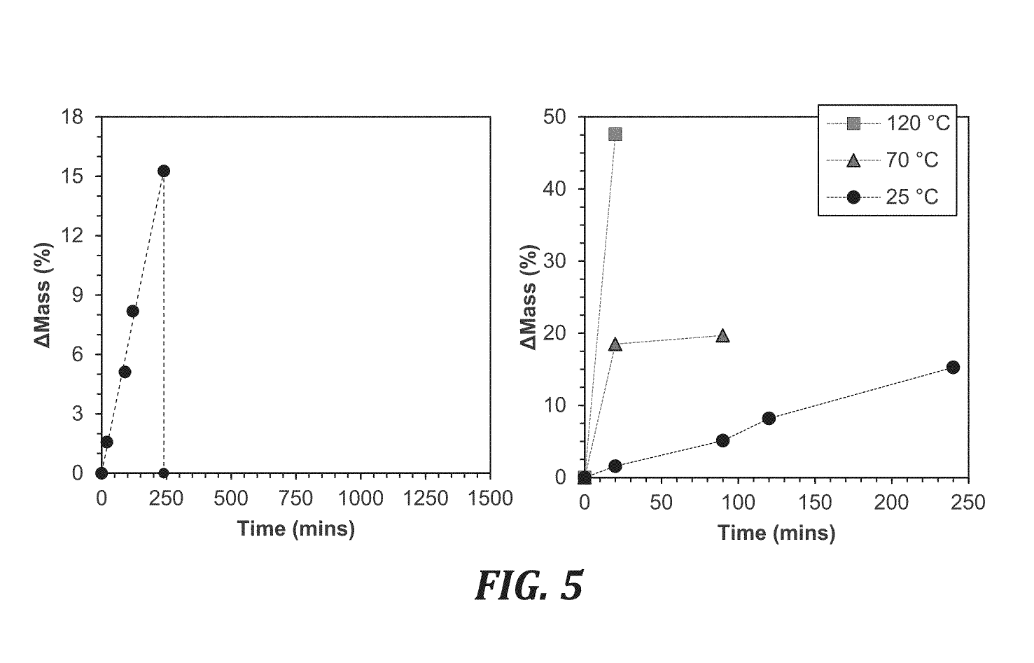

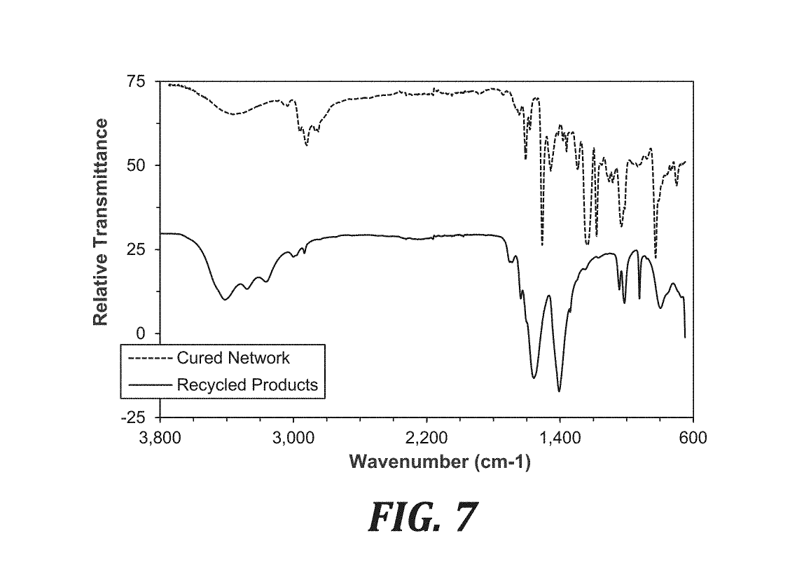

As discussed further herein, the covalently crosslinked polymer networks described herein are configured to degrade under acidic conditions. Degradation of the covalently crosslinked polymer networks described herein can be associated with a decline in the structural integrity of the covalently crosslinked polymer network. In certain embodiments, such a decline in structural integrity can be measured, for example, by cracking and crazing of the covalently crosslinked polymer networks as a result of exposure to acidic conditions, as described further herein with respect to FIG. 5. In certain embodiments, such a decline of structural integrity can also be measured, for example, spectroscopically through the measurement of the breaking of chemical bonds as result of exposure to acidic conditions, as discussed further herein with respect to FIG. 7.

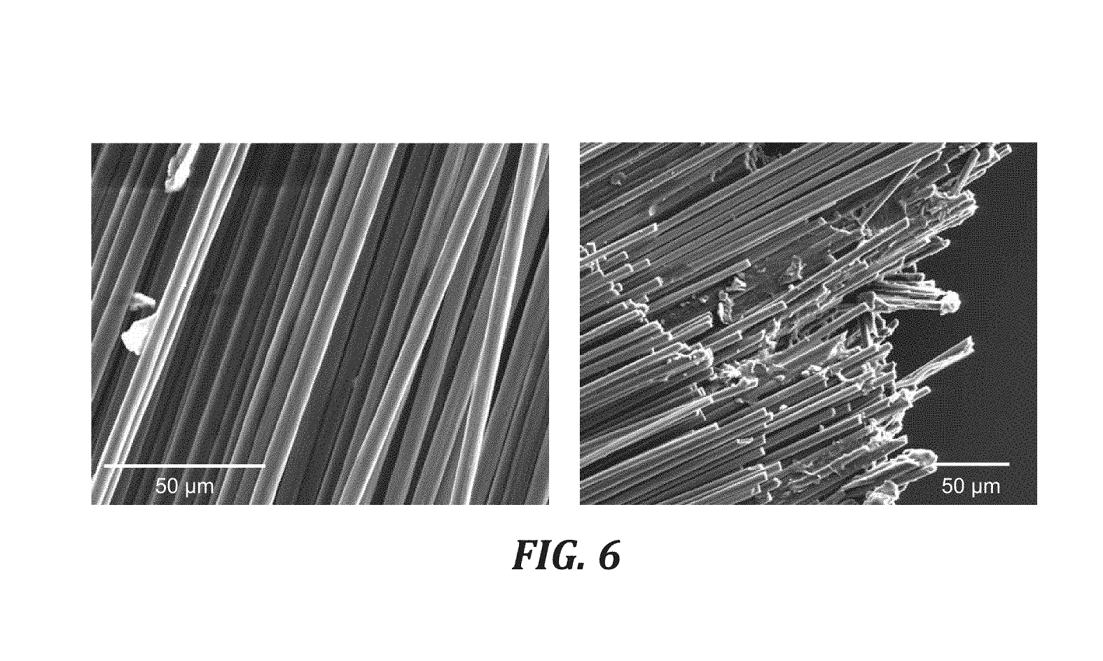

In an embodiment, the covalently crosslinked polymer network is configured degrade in a solution having a pH of about 2.5 or lower. In an embodiment, the covalently crosslinked polymer network is configured degrade in a solution having a pH in a range of about 2.5 to about 1.0. As described further herein, the covalently crosslinked polymer networks degrade in acidic conditions, such as in an acidic solution, such that the covalently crosslinked polymer network breaks into a number of pieces, is converted to a gel, is dissolved in a solution, and/or is dispersed in a suspension, thus exposing fibers that were previously disposed in the covalently crosslinked polymer network. In an embodiment, fibers exposed by acid-based recycling of the covalently crosslinked polymer networks described herein have only negligible differences in length compared to their length prior to incorporation in the covalently crosslinked polymer network, as discussed further herein with respect to FIG. 6.

In an embodiment, the covalently crosslinked polymer network has a glass transition temperature in a range of about 100° C. to about 150° C. In an embodiment, the covalently crosslinked polymer network has a glass transition temperature in a range of about 110° C. to about 140° C. In an embodiment, the covalently crosslinked polymer network has a glass transition temperature in a range of about 110° C. to about 120° C., as described herein with respect to the thermal properties of xTAZ-DGEBA polymer networks. Such relatively high glass transition temperatures are suitable for some conventional manufacturing processes.

In an embodiment, the covalently crosslinked polymer network has a thermal degradation onset temperature in a range about 300° C. to about 400° C. In an embodiment, the covalently crosslinked polymer network has a thermal degradation onset temperature in a range of about 330° C. to about 380° C. In an embodiment, the covalently crosslinked polymer network has a thermal degradation onset temperature between about 340° C. and about 360° C., as described herein with respect to the thermal properties of xTAZ-DGEBA polymer networks.

In an embodiment, the curative agent is soluble in the prepolymer. In this regard, reactions between a curative agent and the prepolymer may be free of a solvent.

In an embodiment, the covalently crosslinked polymer network has a cure conversion greater than 75%. In an embodiment, the covalently crosslinked polymer network has a cure conversion greater than 85%. In an embodiment, the covalently crosslinked polymer network has a cure conversion greater than 95% as described herein with respect to the thermal properties of xTAZ-DGEBA polymer networks.

In an embodiment, the covalently crosslinked polymer network has a storage modulus in a range of about 1,500 MPa to about 2,000 MPa. In an embodiment, the covalently crosslinked polymer network has a storage modulus in a range of about 1,700 MPa to about 1,900 MPa. In an embodiment, the covalently crosslinked polymer network has a storage modulus in a range of about 1,800 MPa to about 1,900 MPa. Such storage moduli are comparable to or better than storage moduli of conventional covalently crosslinked polymer networks.

In another aspect, the present disclosure provides a curative agent including a hexahydrotriazine moiety. As described further herein, the curative agents of the present disclosure are configured to react with a prepolymer to provide a covalently crosslinked polymer network and, in certain embodiments, composites comprising a plurality of fibers.

In an embodiment, the curative agent has the structure:

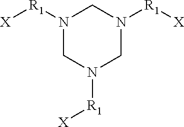

wherein, each R₁ is independently selected from the group consisting of alkyl, alkenyl, alkynl, sulfone, and aryl groups, and each X is independently selected from a hydroxyl group, a primary amine, and a secondary amine.

In an embodiment, the curative agent has the structure:

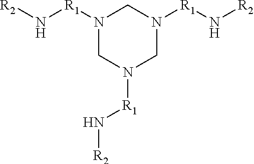

wherein, each R₁ is independently selected from the group consisting of alkyl, alkenyl, alkynl, sulfone, and aryl groups, and each R₂ is independently selected from the group consisting of H, alkyl, alkenyl, alkynl, and aryl groups.

In an embodiment, the curative agent is selected from the group consisting of:

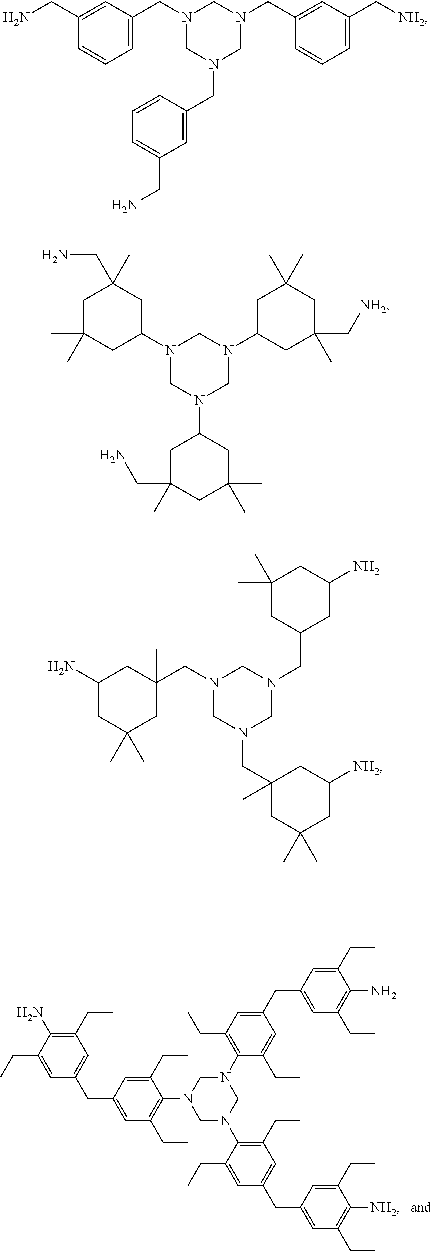

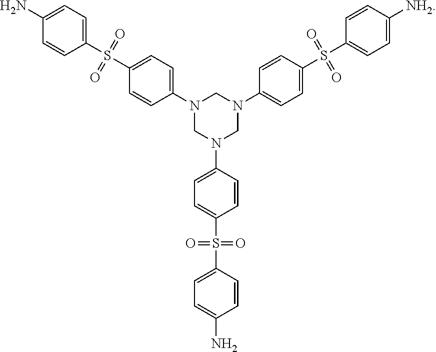 [SMILES: Nc1ccc(S(=O)(=O)c2ccc(N3CN(c4ccc(S(=O)(=O)c5ccc(N)cc5)cc4)CN(c4ccc(S(=O)(=O)c5ccc(N)cc5)cc4)C3)cc2)cc1]

In another aspect, the present disclosure provides a composite comprising a covalently crosslinked polymer network as described herein; and a plurality of fibers disposed within the covalently crosslinked polymer network. In an embodiment, the plurality of fibers include fibers selected from the group consisting of carbon fibers, glass fibers, aramid fibers, hemp fibers, sisal fibers, coconut fibers, and combinations thereof.

In an embodiment, the covalently crosslinked polymer network is recyclable under acidic conditions. In that regard, the covalently crosslinked polymer network breaks down, such as by crazing, cracking, dissolving, and the like to expose the plurality of fibers. Such fibers are then recoverable for later use, such as in a new composite. As discussed further herein, such recovered fibers generally have an average length that is not measurably different than in an initial state before incorporation into the covalently crosslinked polymer network. In an embodiment, the recovered fibers have a weight that is 1% or less heavier than in an initial state before incorporation into the covalently crosslinked polymer network.

In another aspect, the present disclosure provides a method of making a covalently crosslinked polymer network. In an embodiment, the method includes providing a mixture comprising: a curative agent comprising a pH-responsive chemical linkage; and an epoxy; and heating the mixture at a temperature and for a time sufficient to form a covalently crosslinked polymer network that degrades in acidic conditions. In an embodiment, the curative agent comprises a hexahydrotriazine moiety. In an embodiment, the pH-responsive linkage includes the hexahydrazine moiety.

In an embodiment, the curative agent has the structure:

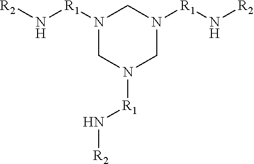

wherein,

each R₁ is independently selected from the group consisting of alkyl, alkenyl, alkynl, sulfone, and aryl groups, and

each R₂ is independently selected from the group consisting of H, alkyl, alkenyl, alkynl, and aryl groups.

In an embodiment, the curative agent has the structure:

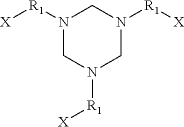

wherein,

each R₁ is independently selected from the group consisting of alkyl, alkenyl, alkynl, sulfone, and aryl groups, and

each X is independently selected from a hydroxyl group and an amine.

In an embodiment, the mixture further comprises a plurality of fibers dispersed therein, and wherein heating the mixture provides a plurality of fibers disposed within the covalently crosslinked polymer network. In this regard, the method is configured to provide a composite as described herein.

In an embodiment, heating the mixture comprises heating the mixture to a temperature in a range of about 100° C. to about 200° C. In an embodiment, heating the mixture comprises heating the mixture to a temperature in a range of about 120° C. to about 180° C. In an embodiment, heating the mixture comprises heating the mixture to a temperature in a range of about 100° C. to about 150° C., as described herein with respect to amine-epoxy network preparation.

The method includes heating the mixture for a time sufficient to form the covalently crosslinked polymer network. In an embodiment, heating the mixture comprises heating the mixture for a time in a range of about 1 hour to about 5 hours. In an embodiment, heating the mixture comprises heating the mixture for a time in a range of about 1 hour to about 3 hours. In an embodiment, heating the mixture comprises heating the mixture for a time in a range of about 1 hour to about 2 hours.

As discussed further herein, in an embodiment the curative network is soluble in the prepolymer. Accordingly, in an embodiment, the mixture does not include a solvent of the curative agent or the prepolymer.

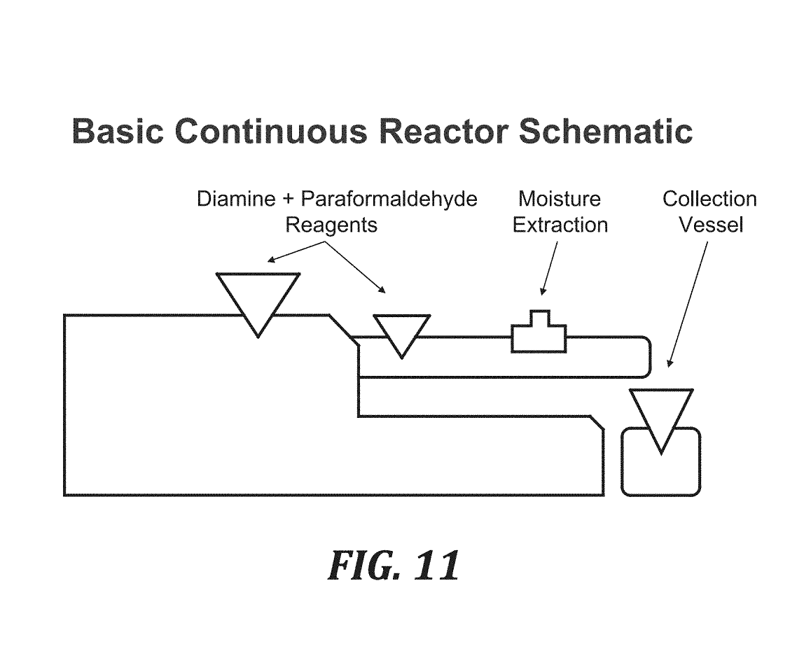

In embodiment, the method of making the covalently crosslinked polymer network is a continuous or semi-continuous process. In an embodiment, the process includes the use of a twin-screw extruder. In such a method, a curative agent including a hexahydrazine moiety is prepared by introducing a di-functional primary amine and paraformaldehyde into a twin-screw extruder. See for example FIG. 11. In the twin-screw extruder, the di-functional primary amine and the paraformaldehyde are mixed under high shear conditions to form a mixture. The shear conditions may be modulated through the number of RPM of the twin-screw extruder. In an embodiment, the mixture is heated, such as in a range of about 100° C. to about 200° C., in various portions of the twin-screw extruder and at various points in the reaction process. By-products of the reaction between the bi-functional amine and paraformaldehyde, such as water, may be removed from the twin-screw extruder through vented sections of the reactor barrel and/or through volatilization at an exit zone of the reactor.

In another aspect, the present disclosure provides a method of recycling a covalently crosslinked polymer network.

In an embodiment, the method of recycling the covalently crosslinked polymer network includes: exposing the covalently crosslinked polymer network to acidic conditions, thereby degrading the covalently crosslinked polymer network, wherein the covalently crosslinked polymer network comprises: a reaction product of a prepolymer and a curative agent comprising a hexahydrotriazine moiety. In an embodiment, the curative agent is any curative agent as described herein. In an embodiment, the prepolymer is any prepolymer described herein. In an embodiment, the covalently crosslinked polymer network is any covalently crosslinked polymer network described herein.

In an embodiment, exposing the covalently crosslinked polymer network to acidic conditions includes contacting the covalently crosslinked polymer network with a solution having a pH of about 2.5 or less, as described herein with respect to network degradation and recycling. In an embodiment, exposing the covalently crosslinked polymer network to acidic conditions includes contacting the covalently crosslinked polymer network with a solution having a pH in a range of about 2.5 to about 1.0. In an embodiment, exposing the covalently crosslinked polymer network to acidic conditions includes contacting the covalently crosslinked polymer network with a solution having a pH configured to degrade the covalently crosslinked polymer network but not configured to degrade any fibers disposed therein, such as a pH in a range of about 2.5 to about 1.0. See, for example, FIGS. 5 and 6 and EXAMPLE 4. In an embodiment, the solution has a temperature of in a range of about 40° C. to about 120° C. In an embodiment, exposing the covalently crosslinked polymer network to acidic conditions converts the covalently crosslinked polymer network from a solid into a gel. See, for example, FIG. 5.

Any acid can be used in exposing the covalently crosslinked polymer network to acidic conditions. In an embodiment, the acidic conditions include an acidic solution. In an embodiment, the acidic conditions include an acidic solution comprising an organic acid. In an embodiment, the acidic conditions include an acidic solution comprising an inorganic acid. In an embodiment, the acidic conditions include an acidic solution comprising an acid selected from the group consisting of acetic acid, hydrochloric acid, sulfuric acid, phosphoric acid, nitric acid, hydrobromic acid, hydroiodic acid, hydrofluoric acid, and combinations thereof.

In an embodiment, exposing the covalently crosslinked polymer network includes processes including spraying, submersing, dip-coating, or otherwise contacting the covalently crosslinked polymer network with an acidic solution, suspension, or slurry.

In an embodiment, the covalently crosslinked polymer network further comprises a plurality of fibers disposed therein. In embodiment, the method includes removing the degraded covalently crosslinked polymer network from the plurality of fibers. In an embodiment, the method of recycling does not substantially affect an average length of the plurality of fibers. See, for example, FIG. 6. In an embodiment, the method includes separating at least some of the plurality of fibers from the covalently crosslinked polymer network.

In an embodiment, recycling includes chemically and/or physically degrading the covalently crosslinked network. In an embodiment, the method includes physically breaking the covalently crosslinked polymer network. In an embodiment, physically breaking the covalently crosslinked network creates a larger surface area exposed acidic conditions. In an embodiment, physically breaking the covalently crosslinked polymer network includes a method selected from the group consisting of crushing, bending, cutting, twisting, tearing, and compressing the covalently crosslinked polymer network, and combinations thereof.

### EXAMPLES

The first epoxy networks containing pH-responsive hexahydrotriazine moieties in the form of an amine curative agent were prepared into recyclable thermoset materials. Recycling was carried out using acetic acid and was shown to occur in less than 24 hours at room temperature with the rate of degradation as a function of temperature being approximately exponential. Analysis of the recycled polymer products revealed a partially soluble material that is hypothesized to be of high molecular weight. Characterization of the recyclate using FTIR revealed high concentrations of amine functional groups that are believed to have formed due to hexahydrotriazine protonation. Further, their comparable thermal and thermomechanical properties indicate that hexahydrotriazine-based amine curative agents are promising as epoxy matrix materials for recyclable composites.

### Example 1

### Curative Synthesis

Materials

Paraformaldehyde (PF, powder, 95%), m-xylyenediamine (MXDA, 99%), glacial acetic acid (1 M, 99.5%), 1-methyl-2-pyrrolidinone (NMP, anhydrous, 99.5%) and deuterated chloroform (CDCl₃, 99.8%) were purchased from Sigma Aldrich Co. and used without purification. Diglycidylether of bisphenol A (DGEBA), EPON 828, with epoxide equivalent weight of 185-192 was purchased from Miller-Stephenson and used without modification.

Synthesis of Reversible Hexahydrotriazine Curative Agent

The 1,3,5-hexahydrotriazine triamine (xTAZ) curative agent was synthesized using MXDA and PF in a single-pot method shown in Scheme 1. First, 8.36 g (0.06 mols) of MXDA and 20 mL of NMP were added to a 100 mL round bottom flask equipped with a magnetic stir rod, heated to 100° C., and stirred at 200 RPM. Paraformaldehyde (1.84 g, 0.06 mols) was separately dissolved into 20 mL NMP and then added slowly by transfer pipette to the heated, open vessel until complete addition. After homogenization the temperature was increased to 120° C. and allowed to react for 12 hours. At completion, products were removed from heat, precipitated into de-ionized water and, and filtered under vacuum on a Buchner funnel. The product was then washed three times with a 1 wt % NaOH solution and another three times with de-ionized water. Finally, the final product was dried in vacuo for 12 hours at 100° C. to produce a yellow, highly viscous liquid.

Notes

Quantities: 0.05-0.2 moles

Temperatures: 25-120° C.

Times: 6-24 hours

Yield: 70-95%

SCHEME 1 schematically illustrates an example synthetic procedure for preparation of xTAZ reversible amine curative agent monomers, in accordance with an embodiment of the disclosure.

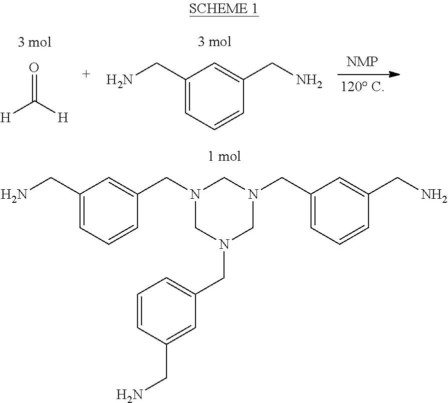 [SMILES: C=O ; NCc1cccc(CN)c1 ; NCc1cccc(CN2CN(Cc3cccc(CN)c3)CN(Cc3cccc(CN)c3)C2)c1]

Preparation of the hexahydrotriazine curative agent monomer, xTAZ, was performed as a one-pot solution synthesis. Due to paraformaldehyde's poor solubility in both polar and non-polar solvents, and its mediocre solubility in xylylene diamine, the synthesis was performed in a strong polar solvent such as NMP to ensure stoichiometric equivalence throughout the reaction medium. At room temperature and through the elevated reaction temperature of 120° C. all reactants were fully soluble.

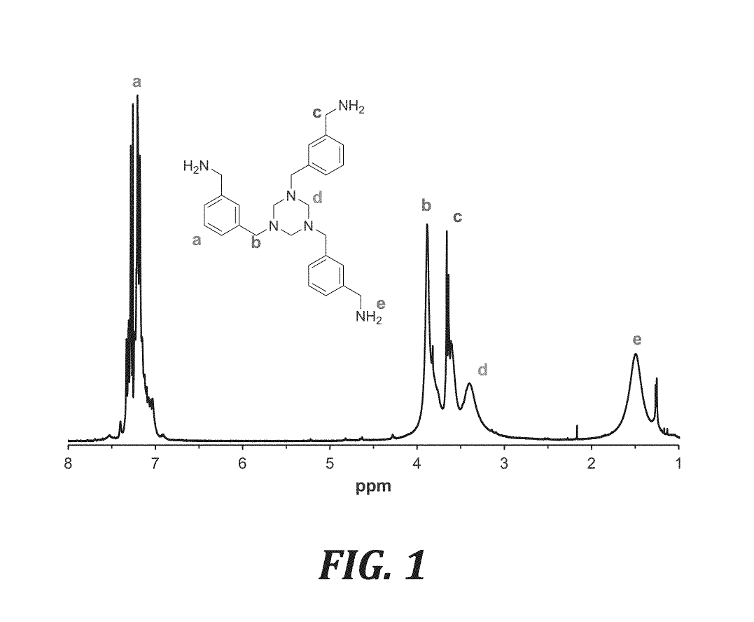

The ¹H-NMR spectrum shown in FIG. 1 reveals the characteristic signals of hexahydrotriazine ring-formation. The three signals between approximately 3 and 4.5 ppm demonstrate three discrete methylene linkages of the amine curative agent, with the peak at 3.35 being characteristic of the hexahydrotriazine group. Quantitative analysis using the aromatic region as an internal standard was performed to characterize the degree of conversion of primary amines into hexahydrotriazine moieties (TABLE 1). Using integration values of the broad methylene triplet (3-4.5 ppm) and the broad primary amine singlet (1.1-2 ppm) reveals a hexahydrotriazine conversion of approximately 94 mol %. The number of methylene protons integrated to 19.03, which corresponds to over one additional proton compared to the theoretical value. Additionally, the calculated number of primary amine protons is under the theoretical value of six. It is hypothesized that the remaining 6 mol % of conversion is related to an intermediate hemiaminal group. As the molecular weights and solubilities of the hexahydrotriazine and hemiaminal molecules are extremely similar, and the hemiaminal intermediates will ring close upon additional heating, no further separation or purification was performed.

TABLE 1
Conversion calculation values from ¹H-NMR

| Integral Values | | | | | | | |
|---|---|---|---|---|---|---|---|
| Ref. | Theoretical | Experimental | Theoretical | Experimental | Conversion Calculation | | |
| Aromatics | CH₂ | CH₂ | NH₂ | NH₂ | CH₂ | NH₂ | Triazine |
| 13.00 | 18.00 | 19.03 | 6.00 | 5.96 | 1.06 | 0.99 | 0.94 |

Transmittance FTIR was performed to verify the NMR characterization and to further understand the chemical nature of the reversible curative. It is well documented that the chemical shift of primary and secondary amines observed during ¹H-NMR characterization can vary due to ionization and broad signals, and therefore, as the nature of the material under consideration is a primary amine curative agent, it was important to confirm the existence of NH₂ functionalities.

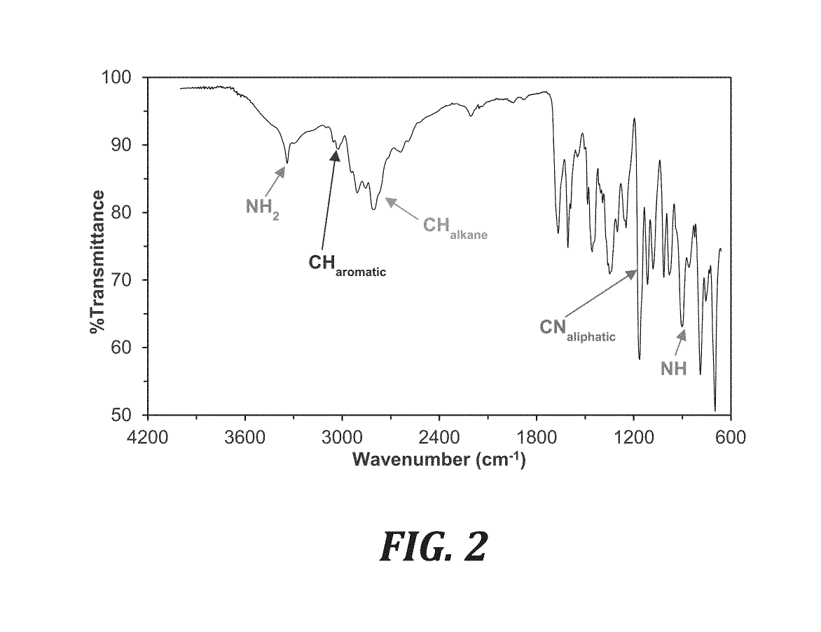

The spectrum in FIG. 2 displays the key functional groups of xTAZ curative agent monomers including primary and secondary amines (800-900 cm⁻¹, 3340 cm⁻¹) and the carbon-nitrogen bonds (1,020, 1,150, 1,160 cm⁻¹) of the hexahydrotriazine. Amino-hydrogen wagging between 800-900 cm⁻¹ reveal two different amine environments due to a small split pattern in the peak. Without wishing to be bound by theory, it is believed that the lower intensity peak at approximately 800 cm⁻¹ is representative of secondary amines found in intermediate hemiaminal groups where the high intensity peak at 900 cm⁻¹ is from the primary amines used for the curing of epoxy networks. The triplet between 1,000-1,200 cm⁻¹ is attributed to stretching of the three distinct carbon-nitrogen bonds within the curative agent: the highest intensity peak for the hexahydrotriazine and the two lower intensity peaks for the aliphatic carbon-nitrogen bonds of MXDA.

Synthesis of Reversible Hexahydrotriazine Curative Agent from 4,4′-Methylenebis (2,6-Diethylanaline)

A 1,3,5-hexahydrotriazine triamine curative agent was synthesized from 4,4′-methylenebis (2,6-diethylanaline) (MBDA) and PF in a single-pot method. First, 0.1 moles of MBDA and 50 mL of NMP were added to a 100 mL round bottom flask equipped with a magnetic stir rod, heated to 100° C., and stirred at 200 RPM. PF (0.1 moles) was separately dissolved into 50 mL NMP and then added slowly by transfer pipette to the heated, open vessel until complete addition. After homogenization the temperature was increased to 150° C. and allowed to react for 12 hours. At completion, products were removed from heat, precipitated into de-ionized water and, and filtered under vacuum on a Buchner funnel. The product was then washed three times with a 1 wt % NaOH solution and another three times with de-ionized water. Finally, the final product was dried in vacuo for 12 hours at 100° C. to produce a cream-yellow solid.

Notes

Reagent Quantities: 0.05-0.2 moles

Reaction Temperatures: 25-180° C.

Reaction Times: 12-24 hours

Yield: 70-99%

Synthesis of Reversible Hexahydrotriazine Curative Agent from 4,4′-Diaminodiphenylsulfone

A 1,3,5-hexahydrotriazine triamine curative agent was synthesized from 4,4′-diaminodiphenylsulfone (DDS) and PF in a single-pot, batch method. First, 0.1 moles of DDS and 50 mL of NMP were added to a 100 mL round bottom flask equipped with a magnetic stir rod, heated to 100° C., and stirred at 200 RPM. PF (0.1 moles) was separately dissolved into 50 mL NMP and then added slowly by transfer pipette to the heated, open vessel until complete addition. After homogenization the temperature was increased to 180° C. and allowed to react for 12 hours. At completion, products were removed from heat, precipitated into de-ionized water and, and filtered under vacuum on a Buchner funnel. The product was then washed three times with a 1 wt % NaOH solution and another three times with de-ionized water. Finally, the final product was dried in vacuo for 12 hours at 100° C. to produce a yellow solid.

Notes

Reagent Quantities: 0.05-0.2 moles

Reaction Temperatures: 25-180° C.

Reaction Times: 12-24 hours

Yield: 70-90%

Synthesis of Reversible Hexahydrotriazine Curative Agents Using Twin-Screw Extruder as a Continuous Reactor

1,3,5-hexahydrotriazine triamine curative agents were synthesized using a continuous reaction process that utilized a co-rotating, intermeshing twin-screw extruder as a high-shear, solvent-less reaction vessel. Base reactants, such as di-functional primary amines and PF in molar ratios of about 1:1, respectively, were used to successfully synthesize and produce 1,3,5-hexahydrotriazine triamines, which were either viscous liquids or solids at room temperature in about 60-300 seconds without need of purification or post-processing. The 1,3,5-hexahydrotriazine triamines were produced in purities of about 70-90% major products.

A 15 mm Labtech twin-screw extruder with 10 temperature-controlled zones was used to synthesize 1,3,5-hexahydrotriazine triamines from a variety of primary diamines, including 1,3-bis(aminomethyl)benzene, 4,4′-methylenebis (2,6-diethylanaline), and 4,4′-diaminodiphenylsulfone. Solid or liquid amines and solid PF were first fed into the feed throat or downstream feed zones of the twin-screw extruder using gravimetric or volumetric powder feeders or peristaltic liquid pumps, respectively, at throughput rates between 1.5 g/minute and 20 g/minute. Barrel sections with feed zones, such as 1-4, were set to temperatures between 25-50° C. to ensure proper feeding into barrel. Subsequent zones, such as 4-8, were set to reaction temperatures between 100-200° C. The final barrel zones, 9-10 were set to temperatures between 50-120° C. to quench the curative as it exited the barrel. Shear states and mixing quality were varied through screw speed which was set between 20 RPM-150 RPM. Water, a by-product formed during the reaction between di-functional primary amines and paraformaldehyde, was removed either through low-pressure vacuum applied to a vented barrel section in either zone 7 or 8, or through volatilization at the reactors exit zone.

### Example 2

### Covalently Crosslinked Network Preparation

Thermoset networks and subsequent test specimens were prepared with DGEBA and either xTAZ or MXDA using a 1:1 stoichiometric equivalence between epoxides and amino-hydrogens. First, reversible curative agent xTAZ was placed in an oven at 100° C. for 10 minutes to lower its viscosity to a workable level. Epoxy and amine curative agents were then mixed at 50° C. for 10 minutes and then cast into preheated silicon molds. The molds were then placed in an oven and held isothermally, in vacuo for 30 minutes at 100° C., ramped to 150° C., and then held again for an additional 1 hour to produce light yellow, glassy epoxy specimens.

### Example 3

### Preparation of Reversible Carbon Fiber Composites

Small composite coupons of reversible epoxy network and carbon fiber were fabricated for use in the degradation study. An aluminum sheet was prepared with Frekote™ sealant and mold releasing agent. The xTAZ and DGEBA network was prepared using the process described in herein above. A chip brush was used to apply the matrix material to two plies of 10 cm×10 cm [0/90] weave. The layup was then vacuum bagged, held under vacuum, and placed into a 100° C. oven. The oven was allowed to equilibrate at temperature and held isothermally for 30 minutes before being ramped to 150° C. Once at the target temperature, the composite was left for one hour before removing from heat and de-bagging.

### Example 4

### Network Degradation and Recycling

Chemical Treatment for Network Recycling

Recyclable epoxy networks and composites were chemically reversed using pH levels <2.5. The thermosets were cured and cut into 15 mm×1 mm cylindrical samples and initial specimen mass was recorded. They were then placed into individual 20 mL scintillation vials with 10 mL of glacial acetic acid, making sure to submerge each in solution. Samples were held at three different temperatures (25, 70, 120° C.) and aliquots were taken at designated time intervals (5, 20, 60, 90, 120, 240, 1440 minutes) to determine relative mass uptake, mass loss, and degradation rate.

Analysis of Network Degradation and Recycling

Recycling of xTAZ-DGEBA epoxy networks was performed by submerging pre-weighed epoxy networks into 1 M glacial acetic acid (pH=2.4). The specimens were then collected at specific time intervals to observe ingress rates, mass uptake, and time to degradation. The findings for the room temperature and elevated temperature experiments are shown in FIG. 5. Room temperature degradation of xTAZ recyclable networks was found to occur rapidly compared to MXDA networks with a relative acid uptake rate of 0.06 g/min and 0.0003 g/min for xTAZ and MXDA, respectively. It was found that after 24 hours MXDA sample masses had only changed approximately 0.6% and therefore data collection was stopped. Conversely, xTAZ networks rapidly uptake solution and display significant visual degradation with a change in mass of over 15% in four hours. At 250 minutes specimen integrity declined due to extreme crazing and stress cracking, thus leading to breakdown into immeasurably small pieces suspended in acetic acid. After 24 hours, the degradation product and acid suspension had separated into two liquid phases, one containing primarily acetic acid and the other recycled epoxy polymer.

Acid ingress experiments were also performed at elevated temperatures to observe the effects of temperature on relative degradation rate (FIG. 5, right). An exponential relationship between mass uptake before degradation and temperature was found. At 70° C. xTAZ networks absorb acetic acid at an approximate rate of 0.93 g/min and degraded into immeasurable pieces at 19 wt %, where recycling at 120° C. led to an approximate ingress rate of 2.38 g/min and uptake at degradation of over 47 wt %. It is important to note that although network breakdown was measured through mass uptake, it is assumed that degradation occurs through multiple mechanisms. Temperature driven rate and ultimate degradation uptake increases are attributed to increased free volume within the network. At 120° C. the networks are at or above T_{g} where free volume has significantly increased, allowing the rubbery network a rapid Fickian ingress response. Further, elevated temperatures are known to increase solvent crazing and stress cracking rates, especially in polymers close to T_{g}. The swelled material eventually fractures under the internal solvent pressure and ultimately leads to surface area growth, further perpetuating the exponential degradation rate.

Additional demonstration of the reversible nature of xTAZ-cured epoxy networks was shown by recycling carbon fiber composite specimens. Two ply coupons were partially submerged in acetic acid for 8 hours to remove the cured matrix material and the fibers were imaged using SEM (FIG. 6). Carbon fibers recycled from xTAZ networks were found to contain minimal residual matrix material after 8 hours of network degradation with the fibers gaining less than 1% of their original mass. Moreover, the change in fiber length was negligible suggesting that xTAZ networks have the ability to maintain neat fiber properties.

Characterization of the degraded network products suggests the recycled material is polymeric in nature and of high molecular weight. The recycled polymer was only partially soluble in THF and CHCl₃ at room temperature but fully soluble at elevated temperatures, suggesting high polymer with some residual gel structure. Chemical analysis and comparison of the xTAZ network and recycled product was then performed using FTIR (FIG. 7). Cured networks were found to contain strong OH stretches (3300 cm⁻¹), aromatic CH stretches (2900 cm⁻¹), and multiple regions of aliphatic CN stretching (1000-1200 cm⁻¹). However, upon acetic acid recycling the strong OH signal becomes covered by NH and NH₂ ovetones ranging from 3100-3500 cm⁻¹, indicating the reversion of hexahydrotriazine rings back into primary and secondary amine functional groups. Additionally, with the degradation of hexahydrotriazine rings, a reduction in the intensity and quantity of CN stretches was found. It is believed that the recycled xTAZ product contains bulky primary amine side groups in the epoxy-based repeat units.

### Example 5

### Thermal Analysis of Recyclable Networks

Differential Scanning Calorimetry (DSC)

Cure information and thermal transitions were obtained using DSC. Both cured and uncured specimens weighing between 5-10 mg were weighed into aluminum pans which were then hermetically sealed. Heat-cool-heat experiments were run using an initial temperature of 25° C., heating and cooling rates of 10° C./minute, and a final temperature of 325° C. Degree of cure was calculated from exothermic peak integrations using Equation (1):

$$α = \frac{Δ {H}_{T} - Δ {H}_{R}}{Δ {H}_{T}} \qquad ( 1 )$$

where a is the degree of cure, ΔH_{T} is the total heat of reaction for an uncured specimen, and ΔH_{R} is the residual heat of reaction for a partially cured specimen.

Thermal Gravimetric Analysis (TGA)

Thermal degradation data was collected via TGA. The cured, 20 mg specimens were placed in platinum pans and ramped from 40-900° C. at 20° C./min to obtain both degradation onset and ultimate char yield information.

Dynamic Mechanical Analysis (DMA)

DMA was conducted on a TA Instruments Q800 equipped with tension clamps to obtain storage and loss modulus data as a function of temperature. Cured, rectangular specimens measuring approximately 30 mm×7.5 mm×1 mm were ramped to 175° C. at a rate of 3° C./min using a constant frequency of 1 Hz and an amplitude of 4 μm.

Scanning Electron Microscopy (SEM) of Recycled Fibers

Carbon fiber specimens before and after recycling were studied via SEM. The samples were rinsed with DI water to remove dust and then dried under vacuum before mounting to SEM posts. Imaging of the fibers was performed using a Vega TS 5136MM instrument set to 15 kV.

Covalently Crosslinked Polymer Network Thermal Properties

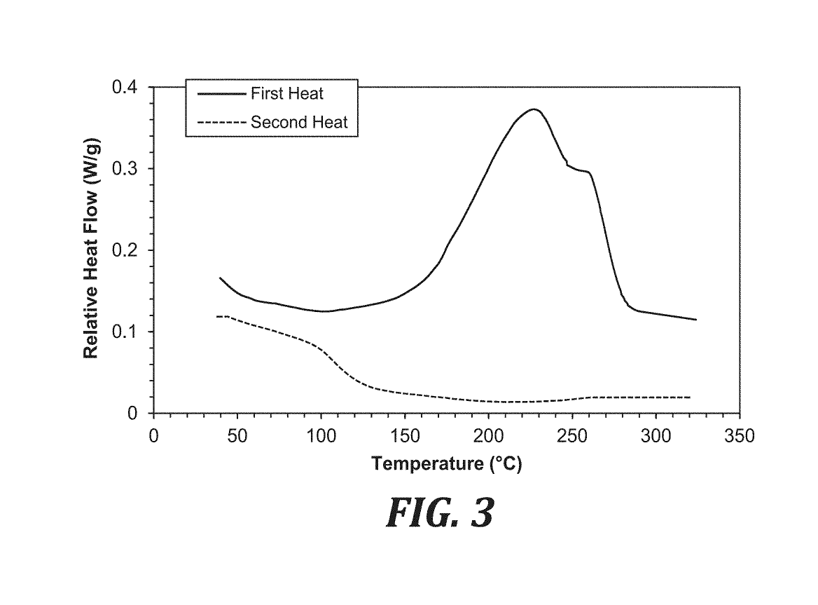

Recyclable thermoset materials such as those studied herein are intended to be drop in replacements for existing, industrially-relevant materials, and as such, principal thermal properties were studied. First, a basic understanding of xTAZ-DGEBA network cure was established via DSC (FIG. 3). The recyclable network exhibits a cure onset temperature of approximately 145° C. and a peak exotherm temperature of 230° C. The shoulder on the exothermic peak at approximately 260° C. is attributed to residual hemiaminal groups gaining enough mobility during cure to ring-close and form hexahydrotriazine.

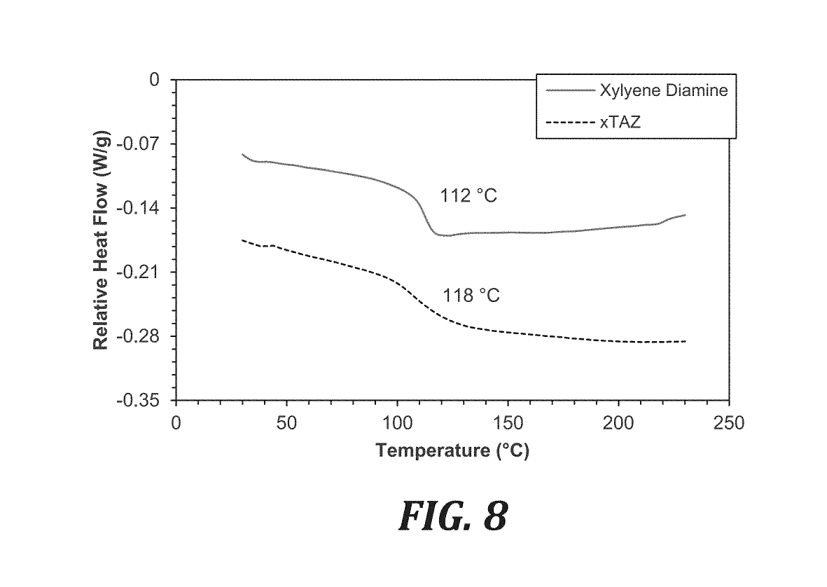

The second heat cycle in FIG. 3 reveals the ultimate glass transition temperature (T_{g}), or the highest achievable T_{g}, after network formation to be between 110-120° C. It is believed the moderate T_{g} of the xTAZ-DGEBA network is caused by the flexible methylene linkages of the MXDA precursor. This was confirmed upon curing DGEBA with MXDA, where both networks have approximately the same glass transition (see, example, FIG. 8). This finding shows that inclusion of the hexahydrotriazine ring did not negatively affect the network's fundamental thermal properties and it is further believed that this trend will continue into other hexahydrotriazine-based amine curative agents. Additionally, inclusion of hexahydrotriazine moieties into the epoxy network did not lead to premature thermal degradation as no secondary exotherm was observed between 150 and 300° C. Further confirmation of the networks thermal stability was measured by TGA. Finally, as the cure onset temperature was 30° C. above the ultimate T_{g}, a cure profile of one hour at 150° C. was found to provide networks with cure conversion greater than 95%; the remaining results are presented using such networks.

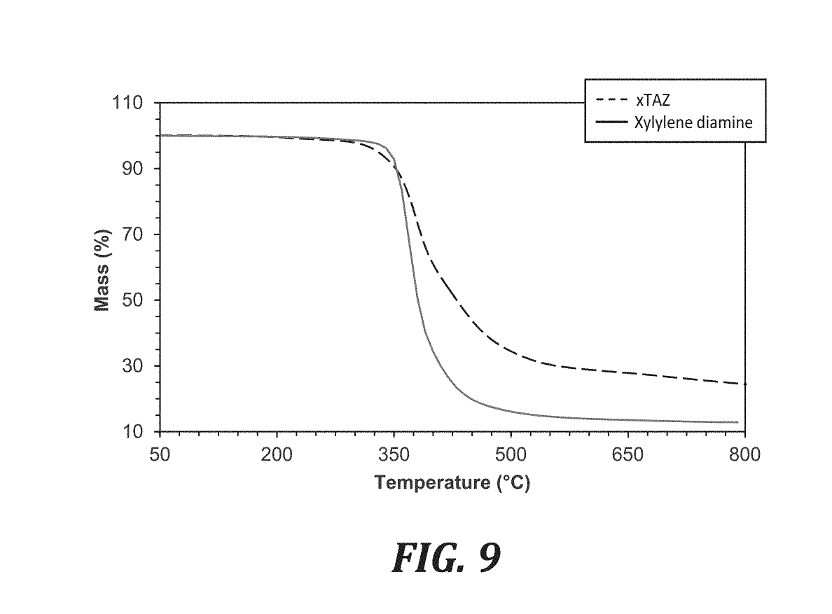

To verify cured network thermal stability, mass loss as a function of temperature was assessed by TGA. The thermogram in FIG. 9 shows a typical, single degradation onset temperature and a 5% mass loss at 330±2° C. The single onset temperature indicates a primary degradation initiation mechanism, a common feature in epoxy networks with high degrees of cure. Upon comparison to DGEBA networks cured using MXDA, it was found that xTAZ network degradation is shifted to a lower temperature of 344±4° C. (see FIG. 9). However, xTAZ recyclable networks retain significantly more mass after degradation with an average char residue of 24±4 wt % while the chemically similar MXDA network had 13±6 wt %. Elevated char residue of xTAZ networks is attributed to the additional nitrogen atoms within hexahydrotriazine groups which have been shown to promote crosslinking reactions during degradation and carbonization.

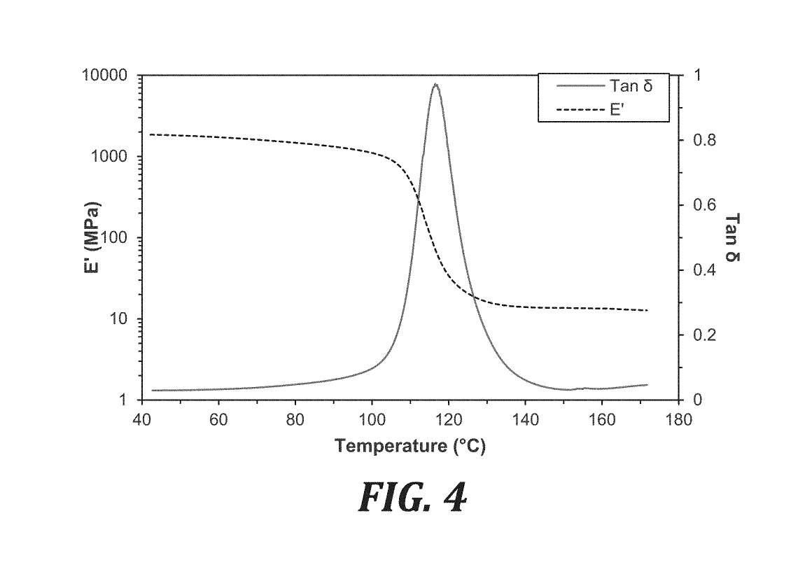

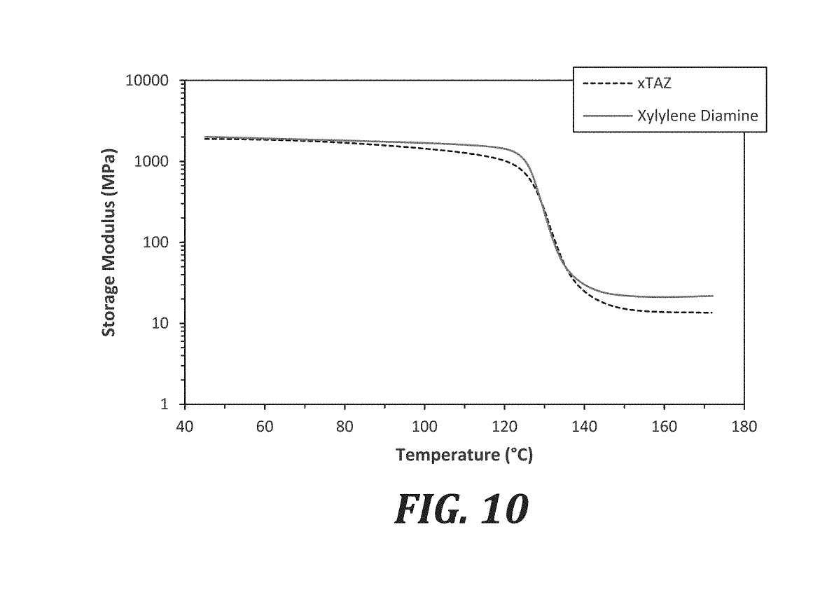

Insight into the recyclable network's thermomechanical properties was gained through DMA (FIG. 4). The material's storage modulus (E′) at 45° C. was found to be over 1850±110 MPa, comparable to MXDA-cured networks (see FIG. 10). The thermomechanical T_{g} was taken from the peak of the tan δ curve and was found to be approximately 116±3° C. The relatively narrow, evenly distributed tan δ curve signifies a homogeneous network and supports the hypothesis that residual hemiaminal groups are converted to hexahydrotriazine during the cure protocol and network formation.

It should be noted that for purposes of this disclosure, terminology such as “upper,” “lower,” “vertical,” “horizontal,” “inwardly,” “outwardly,” “inner,” “outer,” “front,” “rear,” etc., should be construed as descriptive and not limiting the scope of the claimed subject matter. Further, the use of “including,” “comprising,” or “having” and variations thereof herein is meant to encompass the items listed thereafter and equivalents thereof as well as additional items. Unless limited otherwise, the terms “connected,” “coupled,” and “mounted” and variations thereof herein are used broadly and encompass direct and indirect connections, couplings, and mountings. The term “about” means plus or minus 5% of the stated value.

The principles, representative embodiments, and modes of operation of the present disclosure have been described in the foregoing description. However, aspects of the present disclosure which are intended to be protected are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure, as claimed.

1. A kit comprising a curative agent comprising a pH-responsive chemical linkage and a prepolymer, wherein when reacted the curative agent and the prepolymer form a covalently crosslinked polymer network that is degradable in acidic conditions.

2. The kit of claim 1, wherein the curative agent comprises a hexahydrotriazine moiety.

3. The kit of claim 1, wherein the curative agent has the structure: 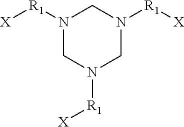
wherein,
each R₁ is independently selected from the group consisting of alkyl, alkenyl, alkynl, sulfone, and aryl groups, and
each X is independently selected from a hydroxyl group, a primary amine, and a secondary amine.

4. The kit of claim 1, wherein the curative agent has the structure: 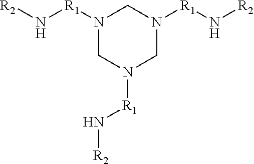
wherein,
each R₁ is independently selected from the group consisting of alkyl, alkenyl, alkynl, sulfone, and aryl groups, and
each R₂ is independently selected from the group consisting of H, alkyl, alkenyl, alkynl, and aryl groups.

5. The kit of claim 1, wherein the curative agent is selected from the group consisting of:

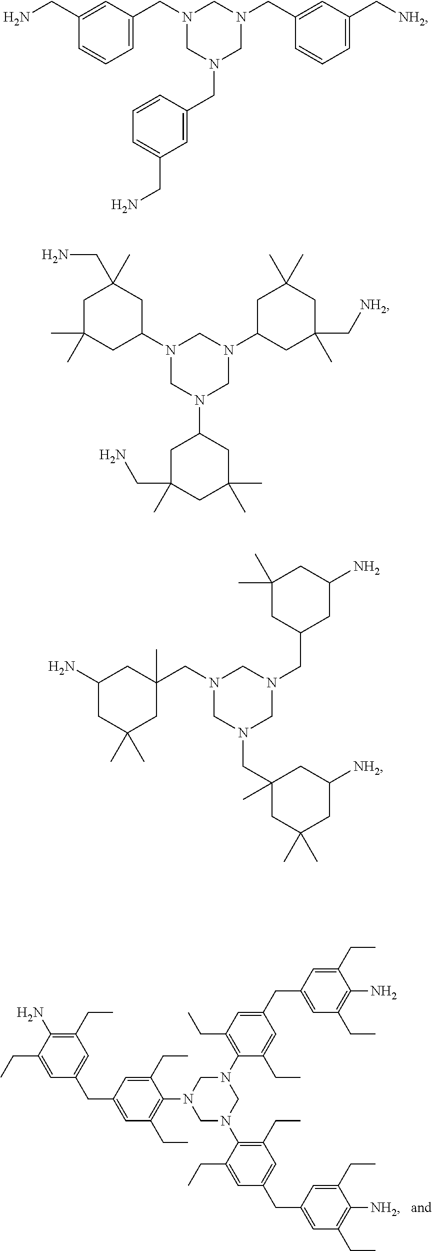

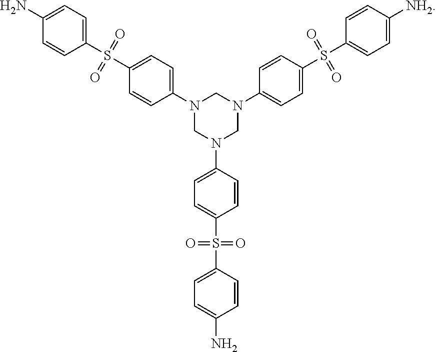 [SMILES: Nc1ccc(S(=O)(=O)c2ccc(N3CN(c4ccc(S(=O)(=O)c5ccc(N)cc5)cc4)CN(c4ccc(S(=O)(=O)c5ccc(N)cc5)cc4)C3)cc2)cc1]

6. The kit of claim 1, wherein the prepolymer comprises an epoxy.

7. The kit of claim 6, wherein the epoxy has the structure: 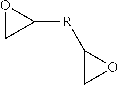
wherein R is selected from the group consisting of alkyl, aryl, alkenyl, and alkynl groups.

8. The kit of claim 6, wherein the epoxy has the structure 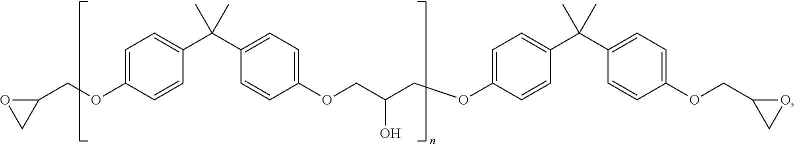
wherein n is an integer between 1 and 5.

9. The kit of claim 6, wherein the epoxy is selected from the group consisting of:

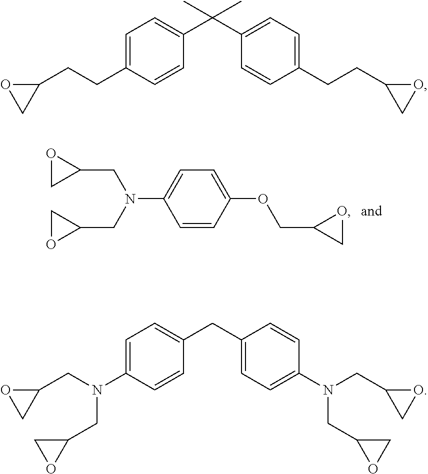 [SMILES: CC(C)(c1ccc(CCC2CO2)cc1)c1ccc(CCC2CO2)cc1 ; c1cc(N(CC2CO2)CC2CO2)ccc1OCC1CO1 ; c1cc(N(CC2CO2)CC2CO2)ccc1Cc1ccc(N(CC2CO2)CC2CO2)cc1]

10. The kit of claim 1, wherein the prepolymer comprises a diisocyanate.

11. The kit of claim 1, wherein the prepolymer comprises a diester.

12. The kit of claim 1, further comprising a plurality of fibers.

13. The kit of claim 12, wherein the plurality of fibers include fibers selected from the group consisting of carbon fibers, glass fibers, aramid fibers, hemp fibers, sisal fibers, coconut fibers, flax fibers, and combinations thereof.

14. A covalently crosslinked polymer network comprising a reaction product of a prepolymer and a curative agent comprising a pH-responsive chemical linkage, wherein the covalently crosslinked polymer network in degradable in acidic conditions.

15-23. (canceled)

24. A composite comprising the covalently crosslinked polymer network of claim 14 and a plurality of fibers disposed within the covalently crosslinked polymer network.

25. (canceled)

26. A method of making a covalently crosslinked polymer network comprising:
providing a mixture comprising:
a curative agent comprising a pH-responsive chemical linkage; and
a prepolymer; and
heating the mixture at a temperature and for a time sufficient to form a covalently crosslinked polymer network degradable under acidic conditions.

27-32. (canceled)

33. A method of recycling a covalently crosslinked polymer network comprising:
exposing the covalently crosslinked polymer network to acidic conditions, thereby degrading the covalently crosslinked polymer network,
wherein the covalently crosslinked polymer network comprises: a reaction product of a prepolymer and a curative agent comprising a pH-responsive linkage.

34-40. (canceled)

41. A curative agent comprising a hexahydrotriazine moiety, wherein the curative agent has the structure: 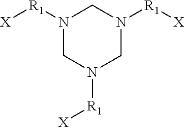
wherein,
each R₁ is independently selected from the group consisting of alkyl, alkenyl, alkynl, sulfone, and aryl groups, and
each X is independently selected from a hydroxyl group, a primary amine, and a secondary amine.

42. (canceled)

43. The curative agent of claim 41, wherein the curative agent has the structure: 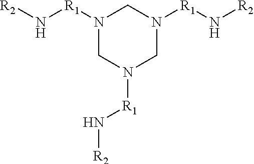
wherein,
each R₁ is independently selected from the group consisting of alkyl, alkenyl, alkynl, sulfone, and aryl groups, and
each R₂ is independently selected from the group consisting of H, alkyl, alkenyl, alkynl, and aryl groups.

44. The curative agent of claim 41, wherein the curative agent is selected from the group consisting of: 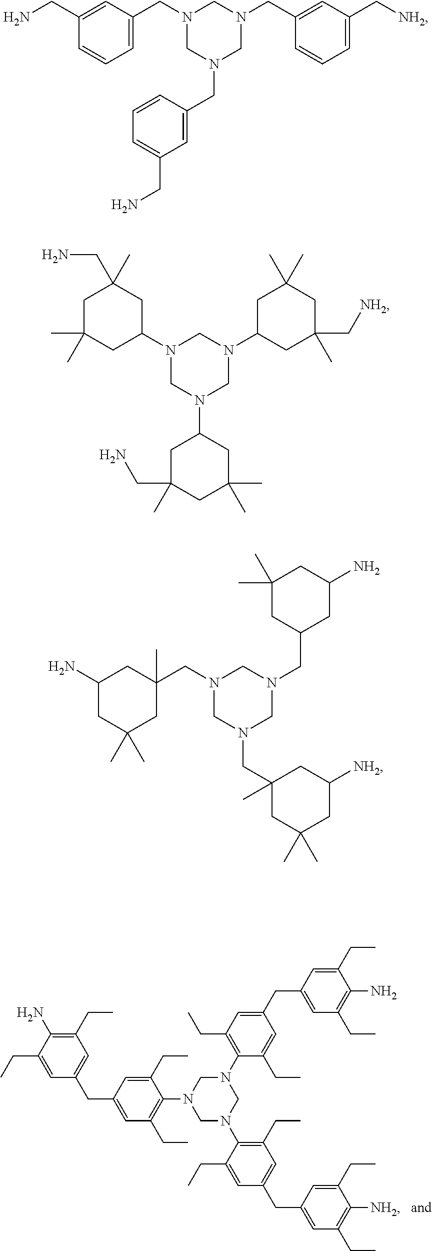 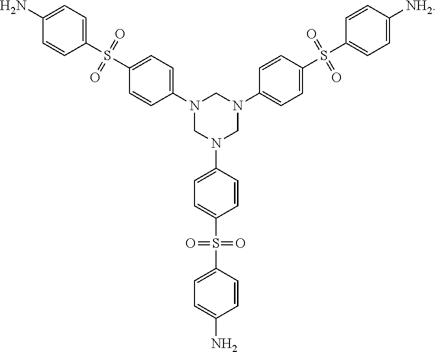 [SMILES: Nc1ccc(S(=O)(=O)c2ccc(N3CN(c4ccc(S(=O)(=O)c5ccc(N)cc5)cc4)CN(c4ccc(S(=O)(=O)c5ccc(N)cc5)cc4)C3)cc2)cc1]
